(19) United States
(12) Patent Application Publication
(10) Pub. No.: US 2018/0310973 A1
(43) Pub. Date: Nov. 1, 2018

(54) ORTHOPEDIC APPARATUS FOR PECTUS EXCAVATUM

(71) Applicants: Seung-Jin Lee, Cheonan-si (KR); CARETECH CO., LTD, Seongnam-si (KR)

(72) Inventors: Yeong-Ho Son, Seongnam-si (KR); Ki-Tae Lee, Seoul (KR); Seung-Jin Lee, Cheonan-si (KR)

(21) Appl. No.: 15/497,942

(22) Filed: Apr. 26, 2017

(51) Int. Cl. A61B 17/80

(52) U.S. Cl. CPC A61B 17/8076; A61B 17/8057

(57) ABSTRACT

Disclosed is an orthopedic apparatus for pectus excavatum, the orthopedic apparatus including: a primary pectus bar inserted behind a concave sternum for pectus deformity repair; a secondary pectus bar inserted in front of the sternum to be in parallel with the primary pectus bar; and a locking member provided at a junction between the primary pectus bar and the secondary pectus bar to lock the primary pectus bar relative to the secondary pectus bar. According to the present invention, the primary pectus bar inserted behind the concave sternum is fixed through the secondary pectus bar that is inserted in front of the sternum, whereby it is possible to prevent the primary pectus bar from rotating or moving during the long period of correction, thereby accurately performing sternum repair.

### BACKGROUND OF THE INVENTION

### Field of the Invention

The present invention relates generally to an orthopedic apparatus for pectus excavatum. More particularly, the present invention relates to an orthopedic apparatus for pectus excavatum for repairing costal cartilage deformity.

### Description of the Related Art

Generally, costal cartilage deformity refers to a symptom of abnormally depressed or protruding sternum, thorax, or ribs.

Particularly, pectus excavatum is a congenital over-depressed deformity of the anterior sternum (thorax or chest wall, hereinafter, referred to as a sternum) and is also referred to as ‘sunken chest’, ‘concave chest’, or ‘funnel chest’. Pectus carinatum is an over-protruding deformity as opposed to pectus excavatum. Pectus excavatum and pectus carinatum may occur independently, but may occur in combination with each other.

Pectus deformity is congenital and generally progresses gradually with age. Although it has no obvious physical symptoms, it is not only bad in appearance but also causes the growth or function of internal organs located in the chest area to deteriorate. Furthermore some patients, especially adolescent patients, may feel difficulty breathing or may suffer fatigue during severe exercise, and they may develop chronic bronchitis or asthma.

Accordingly, it is desirable to treat pectus deformities. In most cases, surgical treatment is needed to treat pectus deformity except in minor cases. Conventional surgical techniques such as sternocostal elevation and sternocostal turnover have been performed. These surgical techniques are performed by opening the entire chest vertically and cutting the sternum, so the operation itself is not only risky for the patient but also leaves a large scar after the operation, whereby patients may not be satisfied, and in severe cases there is a risk of chest instability and sternum necrosis.

To complement the problem of these surgical techniques, in 1998, the Nuss procedure using a metallic bar was introduced by Dr. Donald Nuss. This procedure is a surgical technique where a metallic bar is inserted through the incision of about 2 cm and then is flipped to push out the concave sternum, but it takes long time (approximately several years) to repair the deformity in the state where the pectus bar is fixed in the patient's body. However, compared to the conventional techniques performed by opening the entire chest and cutting the sternum, the Nuss procedure is performed through the minimally invasive incision, whereby the risk of infection is relatively low, the surgical burden is relatively small, scar is minimal, and the patient is satisfied with the time cost. Accordingly, it has been recognized as a safe surgical technique and is a surgical technique now in use.

Because a long period of time is required for sternum repair via the Nuss procedure, the metallic bar should not be repositioned from the initial procedure location inside the patient.

However, although the metallic bar, that is, the pectus bar that substantially performs sternum repair by being inserted behind the sternum should be fixed in place during the long period of time, the position of the pectus bar may be changed or the pectus bar may be rotated. When the procedure location performing repair is changed, accurate sternum repair may not be performed.

Further, the pectus bar requires additional accessories such as a suture and a stabilizer to fix the opposite ends thereof. In the case of suture, it is fixed to the sternum or ribs (the patient's side), so it may cause pneumothorax or hemothorax due to damage of the lung or surrounding blood vessels, and it is difficult for the patient to breathe in the initial stage after the procedure.

The foregoing is intended merely to aid in the understanding of the background of the present invention, and is not intended to mean that the present invention falls within the purview of the related art that is already known to those skilled in the art.

### SUMMARY OF THE INVENTION

Accordingly, the present invention has been made keeping in mind the above problems occurring in the related art, and the present invention is intended to propose an orthopedic apparatus for pectus excavatum, in which a double pectus bar structure configure such that the primary pectus bar is fixed through the secondary pectus bar that is inserted in front of the sternum is applied, whereby the sternum is pushed inward and outward, thereby inducing correction of a shape of the chest wall, by applying the load externally pushed by the primary pectus bar and the load pushed inward by the secondary pectus bar to the chest wall.

The present invention is further intended to propose an orthopedic apparatus for pectus excavatum, in which a structure configure such that the opposite ends of the double pectus bar are fixed to the rib by using a hook-shaped locking means is applied, whereby stable repair of the sternum is possible without patient discomfort.

In order to achieve the above object, according to some aspect of the present invention, there is provided an orthopedic apparatus for pectus excavatum, the orthopedic apparatus including: a primary pectus bar inserted behind a concave sternum for pectus deformity repair; a secondary pectus bar inserted outside the sternum to be in parallel with the primary pectus bar; and a locking member provided at a junction between the primary pectus bar and the secondary pectus bar to lock the primary pectus bar relative to the secondary pectus bar.

The primary pectus bar may be provided with a first coupling hole at each of opposite ends thereof, and the secondary pectus bar may be provided with a second coupling hole at each of opposite ends thereof; and the locking member may be coupled at a location where the first coupling hole and the second coupling hole communicate with each other.

The primary pectus bar may be provided with a first through hole at each of opposite ends thereof.

The primary pectus bar may be provided at each of opposite ends thereof with a notch groove notched toward a center line of the primary pectus bar along a longitudinal direction of the primary pectus bar.

The locking member may include: a locking body screw-engaged with both the first coupling hole and the second coupling hole, and provided with an extended portion at an upper end thereof by extending in a radial direction so as to be stopped by the primary pectus bar; and a locking groove provided by being depressed downward from an upper surface of the locking body at the upper end thereof, and formed in a polygonal shape corresponding to a polygonal ratchet.

The orthopedic apparatus may further include: a stabilizer pressing and supporting the opposite ends of the primary pectus bar so as to prevent the primary pectus bar from rotating in the body.

The stabilizer may be provided at each of the opposite ends of the primary pectus bar and may be perpendicularly coupled to the primary pectus bar.

The stabilizer may include: a connecting body supporting an outer surface of the primary pectus bar by coming into contact therewith; an extended length portion formed by lengthening at each of opposite ends of the connecting body; and a coupling protrusion formed by protruding inward from the extended length portion.

The extended length portion may be provided with a second through hole, and the coupling protrusion may be engaged with the notch groove so as to lock the primary pectus bar.

The primary pectus bar, the secondary pectus bar, and the locking member may be made of a titanium alloy material.

In order to achieve the above object, according to some aspect of the present invention, there is provided an orthopedic apparatus for pectus excavatum, the orthopedic apparatus including: a double pectus bar inserted behind and in front of a sternum to support the concave or convex sternum; and a locking means coupled to each of opposite ends of the double pectus bar and hooked to a rib, on which the opposite ends of the double pectus bar are disposed, to lock the double pectus bar.

The double pectus bar may include: a primary pectus bar inserted behind the sternum, and provided with a first coupling hole at each of opposite ends thereof; a secondary pectus bar inserted outside the sternum to be in parallel with the primary pectus bar, and provided with a second coupling hole at each of opposite ends thereof; and a locking bolt locking the secondary pectus bar to the primary pectus bar by being coupled to both the second coupling hole and the first coupling hole.

The first coupling hole may be in an annular shape with a thread corresponding to a thread of the locking bolt, and the second coupling hole may be in an oblong shape to allow adjustment of a position of the secondary pectus bar relative to the primary pectus bar.

The primary pectus bar may be further provided with a locking hole at each of the opposite ends thereof, with the locking means coupled thereto.

The locking hole may include: an annular first locking hole; and an oblong second locking hole communicating with the first locking hole, and having a width narrower than a diameter of the first locking hole.

The locking means may include: a hook hooked to the rib at a first end thereof; and a fastening bolt engaged with a second end of the hook with a head thereof inserted through the first locking hole, and locked to the hook by the head being moved to the second locking hole.

The locking means may further include: a coupling slide provided with a through slot with the second end of the hook inserted thereinto so as to secure coupling between the hook and the fastening bolt, wherein a state where the second end of the hook is inserted into the through slot is fixed by the fastening bolt.

The hook may be provided with a through hole at the second end thereof, and the coupling slide may be provided with a communicating hole; and the fastening bolt may be engaged with both the communicating hole and the through hole to fix the state where the second end of the hook is inserted into the through slot of the coupling slide.

The hook may be hook-shaped by the first end thereof being bent relative to the second end thereof to be in a C-shape, or may be L-shaped by being bent at 90 degrees.

The double pectus bar and the locking means may be made of a titanium alloy material.

The present invention is advantageous in that the primary pectus bar inserted behind the concave sternum is fixed through the secondary pectus bar that is inserted in front of the sternum, whereby it is possible to prevent the primary pectus bar from rotating or moving during the long period of correction, thereby accurately performing sternum repair.

The present invention is further advantageous in that since the secondary pectus bar firmly fixes the primary pectus bar in place, it is possible to prevent the primary pectus bar from moving or rotating after the procedure. Further, since the locking means fixes the opposite ends of the primary pectus bar by hooking the same to the rib, the primary pectus bar is securely locked and it is possible to push the sternum inside and outside, thereby enabling stable repair of the sternum.

The present invention is further advantageous in that a structure that fixes the opposite ends of the primary pectus bar by hooking the same to the rib does not compress the patient's rib, whereby it is possible to solve the problem that the patient has had difficulty breathing occurring when the conventional suture compresses the rib, and it is possible to facilitate the procedure by simplifying the component configuration.

### BRIEF DESCRIPTION OF THE DRAWINGS

The above and other objects, features and other advantages of the present invention will be more clearly understood from the following detailed description when taken in conjunction with the accompanying drawings, in which:

FIG. 1 shows an exploded perspective view of an orthopedic apparatus for pectus excavatum according to a first embodiment of the present invention;

FIG. 2 shows a front view of a primary pectus bar of the orthopedic apparatus for pectus excavatum according to the first embodiment of the present invention;

FIG. 3 shows a front view of a secondary pectus bar of the orthopedic apparatus for pectus excavatum according to the first embodiment of the present invention;

FIG. 4 shows a front view of a locking member of the orthopedic apparatus for pectus excavatum according to the first embodiment of the present invention;

FIG. 5 shows a front view of a stabilizer of the orthopedic apparatus for pectus excavatum according to the first embodiment of the present invention;

FIGS. 6 to 9 show schematic views of a state where the orthopedic apparatus for pectus excavatum according to the first embodiment of the present invention is inserted into a patient with pectus excavatum;

FIG. 10 shows an exploded perspective view of an orthopedic apparatus for pectus excavatum according to a second embodiment of the present invention;

FIG. 11 shows a combined front view of the orthopedic apparatus for pectus excavatum according to the second embodiment of the present invention;

FIG. 12 shows a combined plane view of the orthopedic apparatus for pectus excavatum according to the second embodiment of the present invention;

FIG. 13A to 13E show exploded perspective views and combined state views of a locking means according to the second embodiment of the present invention;

FIG. 14A to 14C show state views of a process of coupling the locking means according to the second embodiment of the present invention to a double pectus bar;

FIG. 15 shows a view of pectus excavatum (concave chest) of pectus deformity; and

FIG. 16 shows a view of a method of repairing pectus deformity by using the orthopedic apparatus for pectus excavatum according to the second embodiment of the present invention.

### DETAILED DESCRIPTION OF THE INVENTION

Exemplary embodiments of the present invention will be described more fully hereinafter with reference to the accompanying drawings. In the following description of the present invention, detailed descriptions of known functions and components incorporated herein will be omitted when it may make the subject matter of the present invention unclear. The embodiments of the present invention described hereinbelow are provided for allowing those skilled in the art to more clearly comprehend the present invention. Also, for convenience of understanding of the elements, in the figures, sizes or thicknesses may be exaggerated to be large (or thick), may be expressed to be small (or thin) or may be simplified for clarity of illustration, but due to this, the protective scope of the present invention should not be interpreted narrowly.

As described above, the Nuss procedure is a technique in which a pectus bar is inserted through a minimally invasive incision and then is flipped to push out a concave sternum. The Nuss procedure is less expensive than conventional surgical procedures and is recognized as an excellent treatment method.

However, it requires long period for repairing a concave sternum, wherein the pectus bar is fixed in the patient's body for approximately several years. During such a long period, the pectus bar may not be able to perform sternum repair accurately if it is not fixed in place and moves or rotates. In the present invention, two embodiments, i.e. a first embodiment and a second embodiment are proposed as a method of the pectus bar fixation.

### First Embodiment

An orthopedic apparatus for pectus excavatum, which will be described in the first embodiment, is used for repairing thorax deformity, and particularly, used for repairing pectus excavatum (also referred to as funnel chest or concave chest) or costal cartilage deformity.

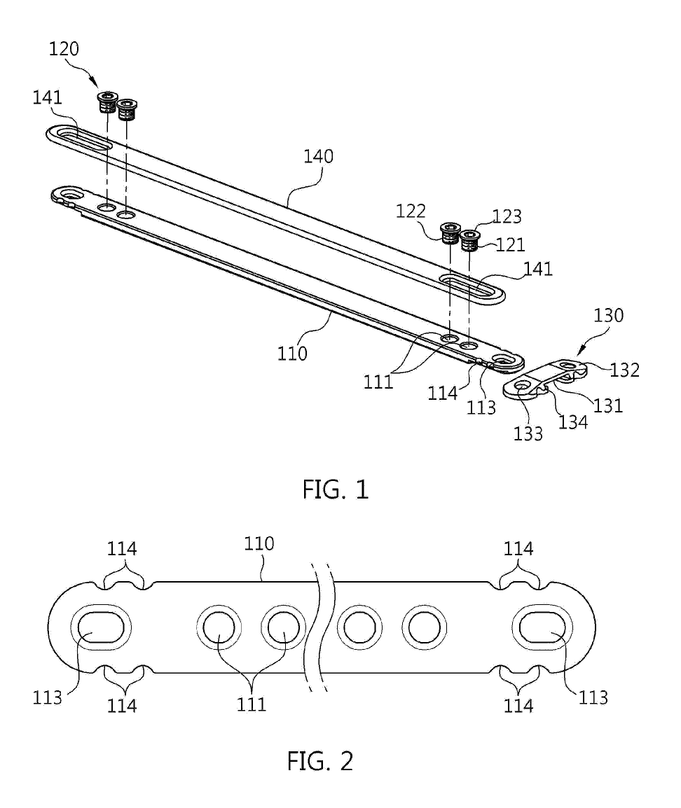

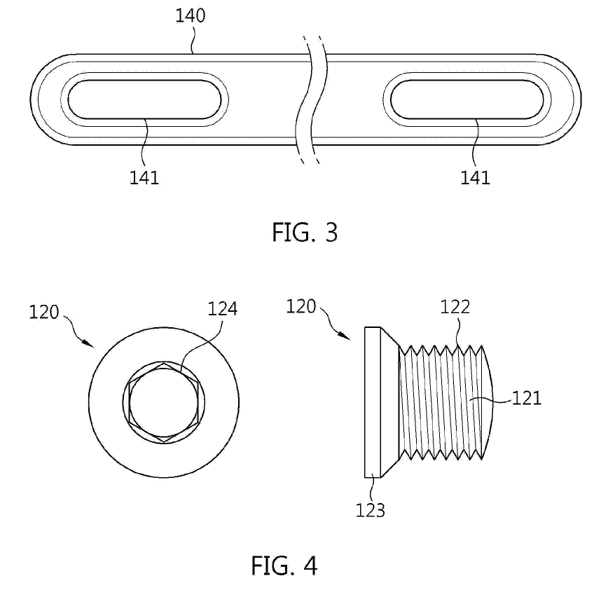

As shown in FIGS. 1 to 5, the orthopedic apparatus for pectus excavatum according to the first embodiment of the present invention includes: a primary pectus bar 110 inserted behind a sternum; a secondary pectus bar 140 inserted in front of the sternum to be in parallel with the primary pectus bar 110; and a locking member 120 provided at a junction between the primary pectus bar 110 and the secondary pectus bar 140 to lock the primary pectus bar 110 to the secondary pectus bar 140.

The primary pectus bar 110 serves to push the sternum from the inside toward the outside of the sternum. In other words, the primary pectus bar serves as a structural support that supports the load of a concave portion, such as a pectus excavatum, behind the sternum. To achieve this, referring primarily to FIGS. 1 and 2, the primary pectus bar 110 is provided in a formed of a bar that is long in a longitudinal direction, and a curved portion is formed at one or multiple parts of the primary pectus bar 110. The curved portion is formed considering a patient's sternum.

Further, since the degree of pectus excavatum differs from patient to patient, the primary pectus bar 110 is configured such that the curvature of the curved portion is changed in response to the concave state. Thereby, it can be applied to asymmetric patients as well as symmetrical patients since a surgeon can form a curvature at any position in the primary pectus bar 110.

The primary pectus bar 110 is provided with a plurality of first coupling holes 111 at opposite ends thereof, the first coupling holes being used to couple the primary pectus bar with the secondary pectus bar 140.

Further, the primary pectus bar 110 is provided with a first through hole 113 at each of opposite ends thereof. A suture is connected to the first through hole 113 such that the primary pectus bar 110 is fixed to the sternum or a rib (the patient's side).

Further, the primary pectus bar 110 is provided at each of opposite ends thereof with a notch groove 114 notched toward a center line of the primary pectus bar 110 along a longitudinal direction of the primary pectus bar 110. The notch groove 114 is engaged with a stabilizer 130 that will be described hereinafter, so as to prevent the primary pectus bar 110 from rotating or moving.

The primary pectus bar 110 configured as described above is disposed behind the sternum to support the concave sternum from behind, and pushes the sternum outward through the curved portion, thereby repairing pectus deformity.

Meanwhile, a length of the primary pectus bar 110 provided in the embodiment varies from 206 mm to 406 mm depending on the patient's body size, such that the surgeon can choose the length to perform a procedure. Further, the primary pectus bar 110 is made of a durable material to prevent deformation caused by the load generated from the concave shape of the sternum.

The secondary pectus bar 140 is provided in parallel with the primary pectus bar 110, and serves to substantially fix the primary pectus bar 110. To achieve this, the secondary pectus bar 140 is inserted over the sternum, and the secondary pectus bar 140 and the primary pectus bar 110 are locked to each other through the locking member 120.

The secondary pectus bar 140 is provided in a form of a bar that is long in a longitudinal direction, like the primary pectus bar 110, and is curved corresponding to the curved shape of the primary pectus bar 110.

The secondary pectus bar 140 is provided with a second coupling hole 141 at each of opposite ends thereof. Further, the locking member 120 is disposed at a location where the first coupling hole 111 and the second coupling hole 141 communicate with each other, to couple the primary pectus bar 110 and the secondary pectus bar 140 together.

The secondary pectus bar 140, in cooperation of the primary pectus bar 110, induces correction of a shape of the chest wall, by applying the load externally pushed by the primary pectus bar 110 and the load pushed inward by the secondary pectus bar 140 to the chest wall.

Thereby, it is possible to prevent a procedure location of the primary pectus bar 110 from being changed during the long period of correction by preventing the primary pectus bar 110 from rotating or moving, thereby accurately performing sternum repair.

Each end of the primary pectus bar 110 and the secondary pectus bar 140 is designed to be curved to minimize damage to soft tissues when they are inserted into the body.

Meanwhile, a length of the secondary pectus bar 140 may be selected from various lengths according to the patient's physical characteristics (such as a width of a thorax), and in the embodiment, the length of the secondary pectus bar 140 varies from 200 mm to 400 mm depending on the patient's body size.

As shown in FIG. 4, the locking member 120 includes: a locking body 121; a locking groove 124 provided by being depressed downward from an upper surface of the locking body 121, namely a ratchet groove 124 in the embodiment; and an extended portion 123 at an upper end of the locking body 121 formed by extending in a radial direction.

The locking body 121 is screw-engaged with both the first coupling hole 111 and the second coupling hole 141, and an outer circumferential surface of the locking body 121 is formed with a male threaded part 122 corresponding to a female threaded part (not shown) that is formed in each of the primary pectus bar 110 and the secondary pectus bar 140, wherein as the male threaded part 122 rotates inward of the female threaded part, coupling force of the secondary pectus bar 140 to the primary pectus bar 110 can be enhanced.

The locking groove 124 is the polygonal ratchet groove 124, and is provided by being depressed downward from the upper surface of the locking body 121 at the upper end thereof In the embodiment, the polygonal ratchet groove 124 is the hexagonal ratchet groove 124 with a hexagonal cross section, and a polygonal ratchet (not shown) is provided in a form of a hexagonal ratchet.

The hexagonal ratchet groove 124 is engaged with the associated hexagonal ratchet, and the associated hexagonal ratchet engaged with the hexagonal ratchet groove 124 rotates the locking body 121, such that the secondary pectus bar 140 is fixedly locked to the primary pectus bar 110.

However, in the embodiment, it is described that the locking groove 124 is provided in the form of the hexagonal ratchet groove 124, but the ratchet groove may be provided in the form of a quadrangular or a star shape, other than the hexagonal shape.

Meanwhile, the locking body 121 is provided with the extended portion 123 at the upper end thereof by extending in the radial direction so as to be engaged with the first coupling hole 111 of the primary pectus bar 110.

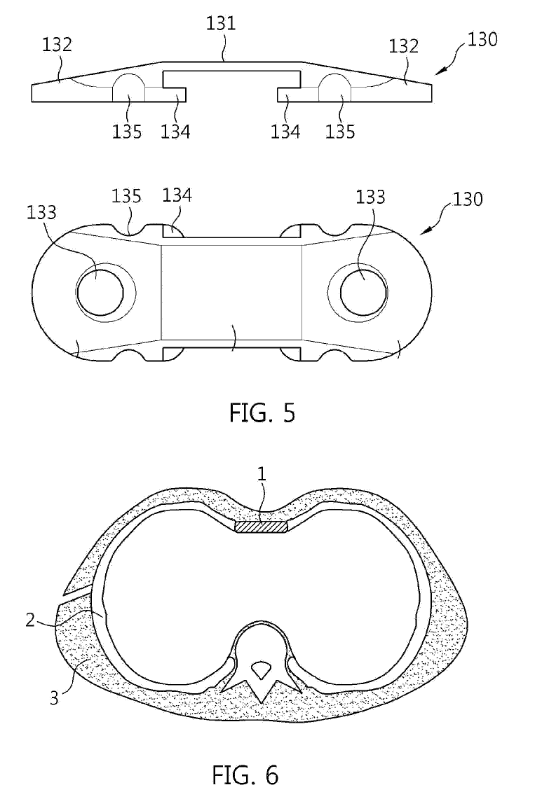

As shown in FIG. 5, the extended portion 123 is formed at the upper end of the locking body 121 by extending in the radial direction, and is integrally formed with the locking body 121.

The extended portion 123 is a portion that is stopped by the primary pectus bar 110, and serves to prevent the locking body 121 from moving inward of the secondary pectus bar 140 or the primary pectus bar 110.

Meanwhile, the primary pectus bar 110 disposed behind the sternum serves to push the concave sternum outward, and by the load applied inward of the concave sternum, a location of the primary pectus bar 110 may be changed from the initial location, or the primary pectus bar maybe twisted to be rotated.

To prevent this, if it is necessary to further fix the primary pectus bar 110 depending on the condition and size of the patient's sternum or the degree of depression of the sternum, the stabilizer 130 that prevents movement and rotation of the primary pectus bar 110 is further provided according to judgment of the surgeon.

Referring to FIG. 5, the stabilizer 130 serves to fix the primary pectus bar 110 by being connected to each of the opposite ends of the primary pectus bar 110, so as to prevent the primary pectus bar 110 from rotating or moving behind the sternum. To achieve this, the stabilizer 130 includes: a connecting body 131 formed to correspond to the cross sectional shape of the primary pectus bar 110, so as to be coupled to the primary pectus bar 110 while surrounding the outer surface thereof; an extended length portion 132 formed by lengthening at each of opposite sides of the connecting body 131; and a coupling protrusion 134 formed by protruding inward from the extended length portion 132 and engaged with the notch groove 114.

The connecting body 131 is provided at each of end portions of the primary pectus bar 110, and supports the end portion of the primary pectus bar 110 by coming into contact therewith. In other words, the connecting body 131 presses the end portion of the primary pectus bar 110 by surrounding the same from the upper surface thereof, thereby preventing the primary pectus bar 110 from moving.

The extended length portion 132 formed by lengthening at each of the opposite sides of the connecting body 131 serves to facilitate coupling between the stabilizer 130 and a rib.

To achieve this, the extended length portion 132 is formed by lengthening from each of the opposite sides of the connecting body 131, and a bottom surface thereof is formed to be flat. Further, the extended length portion 132 is formed in a tapered shape such that a height thereof decreases toward the opposite sides. Thereby, it is possible to minimize damage to soft tissues when it is inserted into the body.

The extended length portion 132 is formed with a plurality of second through holes 133 at a center thereof. Further, the extended length portion 132 is further provided with a plurality of grooves 135. Thereby, a suture is connected to the second through holes 133 or to the grooves 135, in order to fix together the stabilizer 130 and the rib near the patient's armpit.

Further, the extended length portion 132 is provided with the coupling protrusion 134 corresponding to the notch groove 114 of the primary pectus bar 110. The coupling protrusion 134 is engaged with the notch groove 114 (see FIG. 2) to prevent the primary pectus bar 110 from moving or rotating.

Through the stabilizer 130 configured as described above, it is possible to prevent the primary pectus bar 110 from moving or rotating if it is necessary to further fix the primary pectus bar 110 depending on the condition and size of the patient's sternum or the degree of depression of the sternum. Whether the stabilizer 130 is provided or not may be determined according to the condition and the size of the patient's sternum, the degree of depression of the sternum, or the experience and judgment of the surgeon.

Additionally, the size of the stabilizer 130 is minimized, the usability of the procedure is improved, and patient dissatisfaction is minimized in terms of aesthetic appearance after the procedure.

Meanwhile, the primary pectus bar 110, the secondary pectus bar 140, and the locking member 120 are required to be made of a material such as stainless steel or titanium alloy having not only excellent mechanical properties but also excellent biocompatibility. In the embodiment, the material of the primary pectus bar 110, the secondary pectus bar 140, and the locking member 120 is a Ti6l4V ELI material that satisfies the above-mentioned physical properties.

Hereinbelow, the process of procedure of the orthopedic apparatus for pectus excavatum having the above described configuration will be described.

FIGS. 6 to 9 show schematic views of a state where the orthopedic apparatus for pectus excavatum according to the first embodiment of the present invention is inserted into a patient with pectus excavatum.

Firstly, as preparations before use, the apparatus is supplied in a non-sterile condition and should be sterilized by autoclave before the procedure. Further, additional inspection should be involved, such as assessment of cardiac condition and lung function, CT scan, and the like, and an accurate measurement of the surgical site is performed by measuring the thorax and taking photographs so as to determine the length of the bar and the shape to be curved that are appropriate for the degree of deformation and correction.

To briefly describe the process of procedure, firstly, the primary pectus bar 110 appropriate for compression is selected considering the size of the patient's chest and the degree of pectus excavatum. Further, through preoperative examination, the primary pectus bar 110 is curved at a predetermined angle.

Next, as shown in FIG. 6, opposite sides of the thorax are incised to insert the primary pectus bar 110 under the sternum 1.

Next, one end of the primary pectus bar 110 is inserted into one incision and is passed through the other incision. Here, a thoracoscopy is used to confirm that the primary pectus bar 110 passes inside the sternum 1.

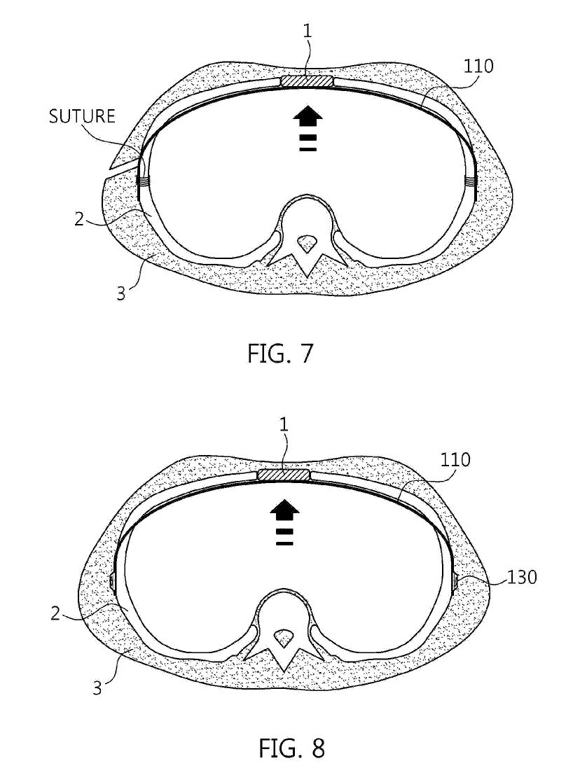

Then, as shown in FIG. 7, after the end of the primary pectus bar 110 is inserted into one incision and is passed through the other incision, it is forcibly flipped to push the sternum 1 up, and then the opposite ends of the primary pectus bar 110 and the rib 2 are fixed together using a suture.

If it is determined that rotation of the primary pectus bar 110 is suspected or a stronger coupling force is needed depending on the size and condition of the patient's sternum 1, as shown in FIG. 8, the stabilizer 130 is inserted into the end of the primary pectus bar 110 that is inserted into under the sternum 1, each of the opposite ends of the primary pectus bar 110 and the stabilizer 130 are coupled together by using the locking member 120, and then the stabilizer 130 is fixed to the rib 2 using a suture.

Here, each end of the primary pectus bar 110 is designed to be curved, and the stabilizer 130 is tapered toward opposite sides thereof, whereby it is possible to minimize damage to soft tissues when they are inserted into the body.

When the stabilizer 130 is used, the primary pectus bar 110 may be stably fixed and adaptation of the primary pectus bar 110 to the growth of the sternum 1 is possible. Since the size of the stabilizer 130 is minimized and procedure can be performed in the state where the stabilizer is attached to the primary pectus bar 110, the usability of the procedure is improved, and patient dissatisfaction is minimized in terms of aesthetic appearance after the procedure.

Then, the secondary pectus bar 140 disposed over the sternum 1 is curved according to the patient's condition. Like the primary pectus bar 110, it may be curved before the procedure and the procedure may be performed.

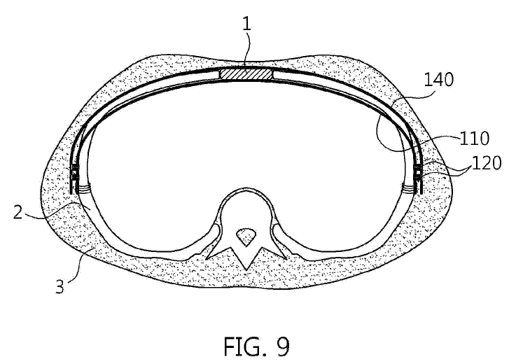

Next, as shown in FIG. 9, after one end of the secondary pectus bar 140 disposed over the sternum 1 is inserted into one incision and is passed through the other incision, it is forcibly flipped to be aligned with the pre-inserted primary pectus bar 110 disposed beneath the sternum 1, and then the locking member 120 is coupled to both the first coupling hole 111 of the inserted primary pectus bar 110 and the second coupling hole 141 of the secondary pectus bar 140.

Finally, if the suture connection between the primary pectus bar 110 and the rib 2 is successful, the locking member 120 is used to confirm that the screw connection between the primary pectus bar 110 and the secondary pectus bar 140 is proper and then the procedure is completed.

As described above, according to the first embodiment of the present invention, since the secondary pectus bar 140 firmly fixes the primary pectus bar 110 in place, it is possible to significantly reduce an effect that the position of the primary pectus bar 110 is changed or the primary pectus bar is rotated, which may occur after the procedure.

Further, the primary pectus bar 110, the secondary pectus bar 140, the stabilizer 130, and the locking member 120 are made of a titanium alloy material to have excellent biocompatibility and mechanical properties.

### Second Embodiment

An orthopedic apparatus for pectus excavatum, which will be described in the first embodiment, is used for repairing sternum and thorax deformity, and particularly, used for repairing pectus excavatum (also referred to as funnel chest), costal cartilage deformity, or pectus carinatum.

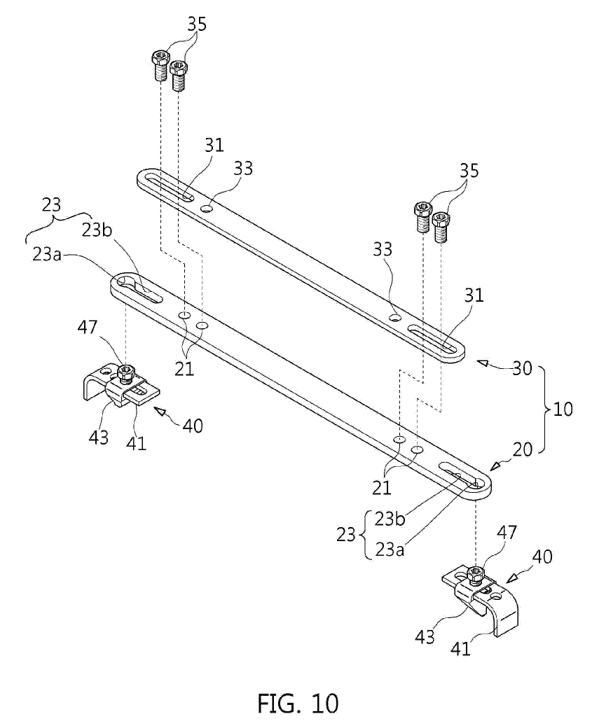

As shown in FIG. 10, the orthopedic apparatus for pectus excavatum according to the second embodiment of the present invention includes: a double pectus bar 10; and a locking means 40.

The double pectus bar 10 supports a concave or convex sternum by being behind inside and in front of the sternum. The double pectus bar 10 enables stable repair of the sternum 1 without rotation or positional change in the body by compressing the sternum 1 internally and externally.

The double pectus bar 10 includes: a primary pectus bar 20; a secondary pectus bar 30; and a locking bolt 35.

The primary pectus bar 20 is inserted behind the sternum 1, and serves to push the sternum 1 outward. The primary pectus bar 20 is provided in a formed of a bar that is long in a longitudinal direction, and is provided with a first coupling hole 21 at each of opposite ends thereof. The first coupling hole 21 is provided two in number at each of the opposite ends of the primary pectus bar, which is used to couple the primary pectus bar with the secondary pectus bar 30.

The primary pectus bar 20 is further provided with a locking hole 23 at each of the opposite ends thereof. The locking means 40 for fixing the opposite ends of the primary pectus bar 20 to a rib is coupled to the locking hole 23.

The locking hole 23 includes: an annular first locking hole 23a; and an oblong second locking hole 23b communicating with the first locking hole 23a, and having a width narrower than a diameter of the first locking hole 23a. The width of the second locking hole 23b corresponds to an external diameter of a thread of a fastening bolt 47. The shapes of the first locking hole 23a and the second locking hole 23b facilitate positioning and locking of the primary pectus bar 20 relative to the locking means 40.

The secondary pectus bar 30 is inserted outside the sternum 1 to be in parallel with the primary pectus bar 20, and serves to push the sternum 1 inward. Further, since the secondary pectus bar 30 prevents the primary pectus bar 20 from rotating or moving, it is possible to prevent a procedure location of the primary pectus bar 20 from being changed during the long period of correction, thereby accurately repairing the sternum 1.

Since the degree of pectus excavatum differs from patient to patient, the secondary pectus bar 30 is configured such that the curvature thereof is changed in response to the concave or convex state to be coupled to the primary pectus bar 20.

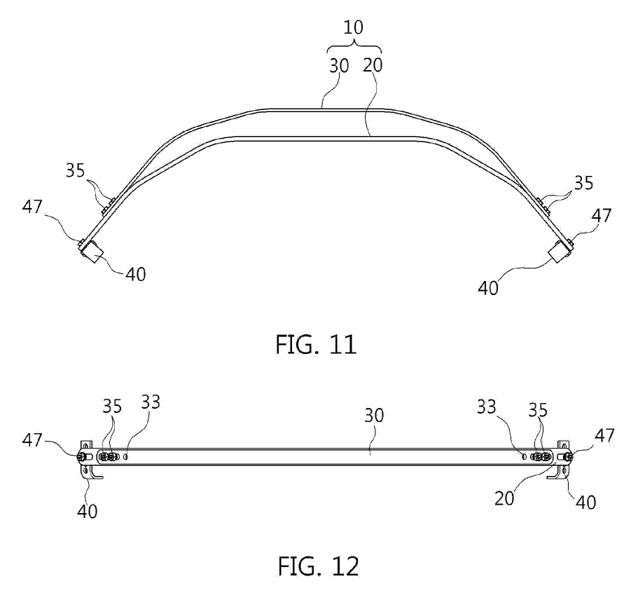

The secondary pectus bar 30 is provided in a form of a bar that is long in a longitudinal direction, and is formed with a second coupling hole 31 at each of opposite ends thereof. The second coupling hole 31 is provided for coupling the secondary pectus bar with the primary pectus bar 20. The second coupling hole 31 is formed in an oblong shape to allow adjustment of a locking position of the secondary pectus bar 30 relative to the primary pectus bar 20, so that the curvature can be changed (see FIG. 11).

The secondary pectus bar 30 is further provided with a through hole 33 at each of the opposite ends thereof. The through hole 33 is a hole that allows a surgical tool to be inserted therethrough to adjust positions of the opposite ends of the primary pectus bar 20 and the secondary pectus bar 30 after the primary pectus bar 20 and the secondary pectus bar 30 are respectively inserted inside and outside the sternum 1 and are coupled to each other.

Widths of the primary pectus bar 20 and the secondary pectus bar 30 are narrower than a width of a gap between ribs. Thereby, it is possible to relieve pain occurring when the widths of the primary pectus bar 20 and the secondary pectus bar 30 are wider than the width of the gap between ribs.

For example, the primary pectus bar 20 may be configured to have a length of 180 m to 280 mm, a width of 10 mm, and a thickness of 2 mm, or be configured to have a length of 300 mm to 400 mm, a width of 12 mm, and a thickness of 2 mm. The difference between widths of the primary pectus bar 20 is intended to increase the area pushing out the sternum in the case of a body with a relatively large sternum.

A length of the secondary pectus bar 30 may vary depending on the patient's physical characteristics. For example, the secondary pectus bar may be configured to have a length of 160 mm to 330 mm, a width of 10 mm, and a thickness of 2 mm.

The lengths, widths, and thicknesses of the primary pectus bar 20 and the secondary pectus bar 30 are configured to facilitate insertion of the primary pectus bar 20 and the secondary pectus bar 30 behind the sternum 1, and to minimize the sensation of a foreign body after insertion

The locking bolt 35 is provided for locking the secondary pectus bar 30 to the primary pectus bar 20.

The locking bolt 35 locks the opposite ends of the secondary pectus bar 30 to the opposite ends of the primary pectus bar 20 by being coupled to both the second coupling hole 31 of the secondary pectus bar 30 and the first coupling hole 21 of the primary pectus bar 20.

The first coupling hole 21 is in an annular shape having a thread corresponding to the thread of the locking bolt 35, and the second coupling hole 31 is in an oblong shape to allow adjustment of a locking position of the secondary pectus bar 30 relative to the primary pectus bar 20.

The secondary pectus bar 30 is shorter than the primary pectus bar 20, and a distance between the second coupling holes 31 formed at the opposite ends of the secondary pectus bar 30 is farther than that between the first coupling holes 21 formed at the opposite ends of the primary pectus bar 20. When the locking bolt 35 is engaged with both the second coupling hole 31 and the first coupling hole 21, the secondary pectus bar 30 has a curved shape with a predetermined curvature with respect to the primary pectus bar 20. The curvature of the secondary pectus bar 30 may be changed according to a locking position where the locking bolt 35 is engaged with the second coupling hole 31.

The locking bolt 35 is screw-engaged with both the second coupling hole 31 and the first coupling hole 21. An outer circumferential surface of the locking bolt 35 is formed with a male threaded part corresponding to a female threaded part that is formed in the first coupling hole 21, wherein as the male threaded part rotates inward of the female threaded part, coupling force of the secondary pectus bar 30 to the primary pectus bar 20 can be enhanced.

A head of the locking bolt 35 is formed in a hexagonal shape so as to be inserted into a surgical tool when tightening or loosening screws. Further, the locking bolt 35 may be configured such that the head thereof is formed with a locking groove by being depressed at a center of an upper surface of the head. The locking groove allows an insertion portion of the surgical tool to be inserted thereinto when the head of the locking bolt 35 is inserted into the surgical tool, thereby facilitating rotation of the locking bolt 35, and thus the secondary pectus bar 30 is easily coupled to the primary pectus bar 20.

The locking means 40 is coupled to each of the opposite ends of the double pectus bar 10, and is fixed to a rib where the opposite ends of the double pectus bar 10 are placed, thereby fixing the double pectus bar 10.

To be more specific, the locking means 40 is coupled to the opposite ends of the primary pectus bar 20 with the secondary pectus bar 30 coupled thereto, so as to fix the opposite ends of the primary pectus bar 20 to the rib 5 by hooking the opposite ends of the primary pectus bar to the rib.

The locking means 40 includes: a hook 41, a coupling slide 43, and a fastening bolt 47.

The hook 41 is hooked to the rib 5 at a first end thereof, and is coupled to the coupling slide 43 at a second end thereof.

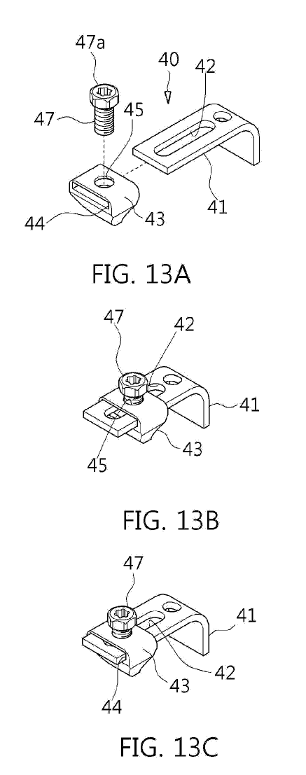

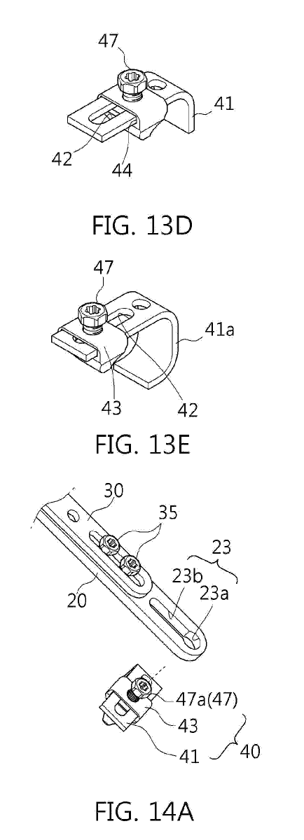

The hook 41 may be hook-shaped by the first end thereof being bent relative to the second end thereof to be in a C-shape (see FIG. 13E), or may be L-shaped by being bent at 90 degrees (see FIG. 13D). The hook 41 is manufactured in two types in order to solve the problem that the hook 41 is not inserted properly due to the soft tissues.

The hook 41 is provided with a through hole 42 at the second end thereof that is coupled to the coupling slide 43. The through hole 42 is engaged with the fastening bolt 47 that is for locking the hook 41 to the coupling slide 43. The through hole 42 is in an oblong shape to allow adjustment of a locking position between the hook 41 and the coupling slide 43.

The coupling slide 43 is provided for securing coupling between the hook 41 and the fastening bolt 47. The coupling slide 43 is formed with a through slot 44 that allows the second end of the hook 41 to be inserted thereinto to be movable, and is further formed with a communicating hole 45 communicating with the through slot 44. The communicating hole 45 is formed to vertically communicate with the through slot 44. The communicating hole 45 is formed at the bottom, such that the fastening bolt 47 engaged through the upper communicating hole 45 may be engaged through to the lower communicating hole (not shown).

An upper surface of the coupling slide 43, which is formed with the through slot 44, is formed to be flat. Thereby, when the fastening bolt 47 engaged with the primary pectus bar 20 is rotated, clamping force is enhanced by the coupling slide 43 disposed between the hook 41 and the primary pectus bar 20, whereby the hook is more securely engaged with the fastening bolt.

The coupling slide 43 is configured such that a lower portion thereof extending downward has a length corresponding to a length of the first end of the hook 41. The lower portion of the coupling slide 43 serves to stably maintain a state where the hook 41 is hooked to the rib 5 by supporting the state.

In other words, in a state where the first end of the hook 41 is hooked to the rib 5, the rib 5 is disposed between the first end of the hook 41 and the lower portion of the coupling slide 43, such that the state where the hook 41 is hooked to the rib 5 is stably fixed.

The fastening bolt 47 is engaged with both the communicating hole 45 and the through hole 42, thereby fixing the state where the second end of the hook 41 is coupled to the coupling slide 43. Further, the fastening bolt 47 is configured such that a head 47a thereof is engaged with the locking hole 23 of the primary pectus bar 20, thereby securely fastening the hook 41 coupled to the coupling slide 43 with the primary pectus bar 20.

The fastening bolt 47 is screw-engaged with both the communicating hole 45 and the through hole 42, and the outer circumferential surface of the locking bolt 35 is formed with a male threaded part corresponding to a female threaded part that is formed in the communicating hole 45. As the male threaded part rotates inward of the female threaded part, coupling force of the hook 41 to the coupling slide 43 can be enhanced.

The fastening bolt 47 may be configured such that the head 47a thereof is in a hexagonal shape, and the upper surface of the head 47a is formed with a locking groove to allow a surgical tool to be inserted thereinto, thereby facilitating rotation of the fastening bolt 47. The locking groove is formed to be a shape corresponding to the insertion portion of the surgical tool.

The locking means 40 may be separately manufactured and then coupled to the primary pectus bar 20 to allow adjustment of a direction of the hook 41 in response to the position of the rib 5.

The primary pectus bar 20, the secondary pectus bar 30, and the locking means 40 may be made of a material having not only excellent mechanical properties but also excellent biocompatibility. For example, they may be made of a titanium alloy material.

The combined state of the orthopedic apparatus for pectus excavatum is shown in FIGS. 11 and 12.

As shown in FIGS. 11 and 12, the orthopedic apparatus for pectus excavatum is configured such that the locking bolt 35 is engaged with both the second coupling hole 31 of the secondary pectus bar 30 and the first coupling hole 21 of the primary pectus bar 20, and the secondary pectus bar 30 has a curved shape with a predetermined curvature with respect to the primary pectus bar 20.

The curvature of the secondary pectus bar 30 with respect to the primary pectus bar 20 may be changed by shifting the coupling position of the locking bolt 35 relative to the oblong second coupling hole 31 in the longitudinal direction.

The opposite ends of the primary pectus bar 20 coupled with the secondary pectus bar 30 may be fixed to the rib 5 by using the locking means 40 having the hook 41.

The locking means 40 may allow adjustment of the curvature of the primary pectus bar 20 in such a way that the head 47a of the fastening bolt 47 is engaged with the locking hole 23 of the primary pectus bar 20, and may also firmly fix the opposite ends of the primary pectus bar 20 to the rib 5.

The locking means 40 is hooked to the rib 5, at which the opposite ends of the primary pectus bar 20 is to be placed in advance, and then is coupled to each of the opposite ends of the primary pectus bar 20.

As shown in FIG. 13A, the second end of the hook 41 is inserted into the through slot 44 of the coupling slide 43, and the fastening bolt 47 is engaged with both the communicating hole 45 of the coupling slide 43 and the through hole 42 of the hook 41.

Here, in a state where the head 47a of the fastening bolt 47 is held in a temporarily coupled state protruding from the upper surface of the coupling slide 43 to a predetermined height, the first end of the hook 41 is hooked to the rib 5.

As shown in FIGS. 13B and 13C, the coupling slide 43 may fixing the rib 5 between the first end of the hook 41 and the coupling slide 43 by shifting the hook 41 toward the first end or the second end thereof.

In this state, when the fastening bolt 47 is further rotated in the tightening direction, as shown in FIG. 13C, the fastening bolt 47 is engaged with the lower communicating hole 45 of the coupling slide 43, and the rib 5 is placed between the first end of the hook 41 and the coupling slide 43, whereby it is possible to firmly fix the state where the first end of the hook 41 is hooked to the rib 5.

In this process, the through hole 42 formed on the second end of the hook 41 is formed to be in an oblong shape, whereby the hook 41 coupled to the coupling slide 43 can be slidingly moved.

The locking means 40 is coupled to the primary pectus bar 20 using the fastening bolt 47 in a state where the coupling slide 43 is coupled to the hook 41.

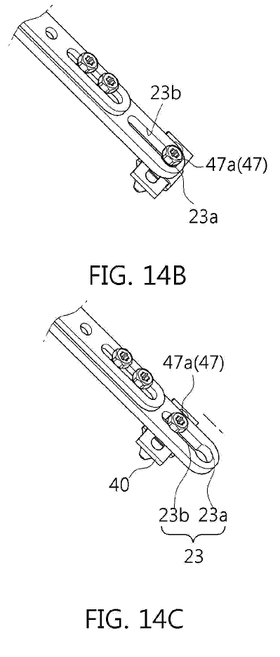

As shown in FIGS. 14A and 14B, the head 47a of the fastening bolt 47 is inserted through the first locking hole 23a of the primary pectus bar 20, and the head 47a of the fastening bolt 47 is moved to the second locking hole 23b to be locked thereto.

In a state where the head 47a of the fastening bolt 47 is moved to the second locking hole 23b, when the fastening bolt 47 is further rotated, the opposite ends of the primary pectus bar 20 are locked by the hook 41 that is hooked to the rib 5, whereby rotation of the hook 41 is prevented, and it is possible to stably repair the sternum 1.

Hereinbelow, the process of procedure of the orthopedic apparatus for pectus excavatum according to the second embodiment of the present invention will be described.

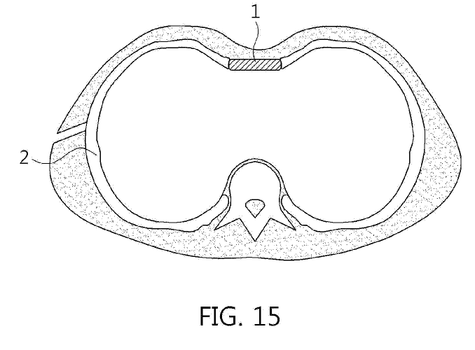

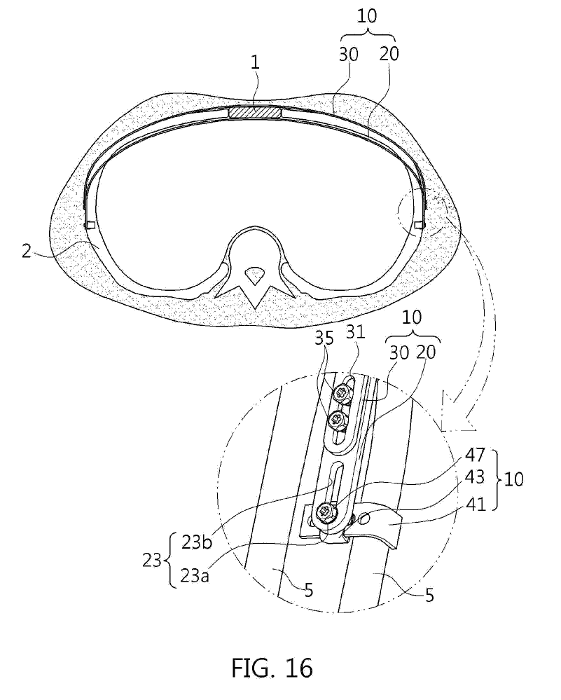

FIG. 15 shows a view of pectus excavatum (concave chest) of pectus deformity; and FIG. 16 shows a view of a method of repairing pectus deformity by using the orthopedic apparatus for pectus excavatum according to the second embodiment of the present invention.

As preparations before use, the orthopedic apparatus for pectus excavatum is supplied in a non-sterile condition and should be sterilized by autoclave before the procedure. Further, additional inspection should be involved, such as assessment of cardiac condition and lung function, CT scan, and the like, and an accurate measurement of the surgical site is performed by measuring the thorax and taking photographs so as to determine the shapes of the primary pectus bar and the secondary pectus bar that are appropriate for the degree of deformation and correction.

Next, as shown in FIG. 15, opposite sides of the thorax are incised about 2 cm to insert the primary pectus bar 20 under the sternum 1.

Next, a first end of the primary pectus bar 20 is inserted into one incision of the thorax, such that a second end thereof is disposed at the other incision. Here, thoracoscopy or endoscopy is used to confirm that the primary pectus bar 20 passes inside the sternum 1.

After the primary pectus bar 20 is forcibly flipped to push the sternum 1 out, the opposite ends of the primary pectus bar 20 is temporarily fixed by using the surgical tool.

Next, a first end of the secondary pectus bar 30 is inserted into one incision, such that the secondary pectus bar 30 is inserted in front of the sternum to be in parallel with the primary pectus bar 20.

Next, the opposite ends of the secondary pectus bar 30 are aligned with the opposite ends of the pre-inserted primary pectus bar 20, and then the locking bolt 35 is engaged with both the first coupling hole 21 of the primary pectus bar 20 and the second coupling hole 31 of the secondary pectus bar 30.

In other words, the locking bolt 35 is engaged with both the second coupling hole 31 formed at the first end of the secondary pectus bar 30 and the first coupling hole 21 formed at the first end of the primary pectus bar 20, and the locking bolt 35 is engaged with both the second coupling hole 31 formed at the second end of the secondary pectus bar 30 and the first coupling hole 21 formed at the second end of the primary pectus bar 20, whereby the opposite ends of the secondary pectus bar 30 are locked to the opposite ends of the primary pectus bar 20.

Here, the curvature of the secondary pectus bar 30 relative to the primary pectus bar 20 may be changed by adjusting a position where the locking bolt 35 is engaged with the second coupling hole 31. In other words, the curvature of the secondary pectus bar 30 relative to the primary pectus bar 20 may be changed in response to the degree of pectus deformity of the patient.

After the curvature of the secondary pectus bar 30 is changed by adjusting a locking position of each of the opposite ends of the secondary pectus bar 30 relative to the primary pectus bar 20, the secondary pectus bar 30 is more securely locked to the primary pectus bar 20 by further rotating the locking bolt 35 to increase clamping force.

Next, each of the opposite ends of the primary pectus bar 20 is coupled to the locking means 40 that is hooked to the rib 5 in advance.

To be more specific, the head 47a of the fastening bolt 47 of the locking means 40 is inserted into the first locking hole 23a formed at the first end of the primary pectus bar 20, the head 47a of the fastening bolt 47 is moved to the second locking hole 23b to correspond to the curvature of the primary pectus bar 20, and then the fastening bolt 47 is further rotated to increase clamping force, whereby the first end of the primary pectus bar 20 is securely locked to the locking means 40 that is hooked to the rib 5.

Next, when the second end of the primary pectus bar 20 is coupled to the locking means 40 that is hooked to the rib 5 in advance through the same manner that is described above, as shown in FIG. 16, the procedure is completed.

When the procedure is completed, whether the coupling between the locking bolt 35 and the fastening bolt 47 is successful is checked, then whether the position where the primary pectus bar 20 and the secondary pectus bar 30 push the sternum 1 outward and inward is appropriate is checked, and then the surgical tool is removed and the incision is closed.

As described above, according to the embodiment of the present invention, since the secondary pectus bar 30 firmly fixes the primary pectus bar 20 in place, it is possible to prevent the primary pectus bar 20 from moving or rotating after the procedure. Further, since the locking means 40 fixes the opposite ends of the primary pectus bar 20 by hooking the same to the rib, the primary pectus bar 20 is securely locked and it is possible to push the sternum inside and outside, thereby enabling stable repair of the sternum.

As described above, according to the second embodiment of the present invention, a double pectus bar structure configure such that the primary pectus bar 20 is fixed through the secondary pectus bar 30 that is inserted in front of the sternum 1 is applied, whereby the sternum 1 is pushed inward and outward, thereby enabling stable repair of the sternum. Further, since the procedure is possible with a procedure time of about 1 hour and the procedure is simple and requires small incisions, the risk of infection is low and there is minimal scarring.

Further, since the locking means 40 fixes the opposite ends of the primary pectus bar 20 by hooking the same to the rib, stable repair of the sternum is possible without patient discomfort.

Meanwhile, the locking means applied in the second embodiment of the present invention is coupled to each of the opposite ends of the double pectus bar for pectus deformity repair and is fixed by being hooked to the rib, but it does not necessarily apply to the double pectus bar but may also be applied to other procedures where the opposite ends need to be fixed by being hooked to the rib.

While the preferred embodiments of the present invention have been described, it is to be understood that the invention is not limited in its application to the details of construction and arrangement of parts illustrated in the accompanying drawings, since the invention is capable of other embodiments and of being practiced or carried out in various ways. Also it is to be understood that the phraseology or terminology employed herein is for the purpose of description and not of limitation.

What is claimed is:

1. An orthopedic apparatus for pectus excavatum, the orthopedic apparatus comprising:
a primary pectus bar inserted behind a concave sternum for pectus deformity repair;
a secondary pectus bar inserted in front of the sternum to be in parallel with the primary pectus bar; and
a locking member provided at a junction between the primary pectus bar and the secondary pectus bar to lock the primary pectus bar relative to the secondary pectus bar.

2. The orthopedic apparatus of claim 1, wherein
the primary pectus bar is provided with a first coupling hole at each of opposite ends thereof, and the secondary pectus bar is provided with a second coupling hole at each of opposite ends thereof; and
the locking member is coupled at a location where the first coupling hole and the second coupling hole communicate with each other.

3. The orthopedic apparatus of claim 1, wherein
the primary pectus bar is provided with a first through hole at each of opposite ends thereof

4. The orthopedic apparatus of claim 1, wherein
the primary pectus bar is provided at each of opposite ends thereof with a notch groove notched toward a center line of the primary pectus bar along a longitudinal direction of the primary pectus bar.

5. The orthopedic apparatus of claim 2, wherein the locking member includes:
a locking body screw-engaged with both the first coupling hole and the second coupling hole, and provided with an extended portion at an upper end thereof by extending in a radial direction so as to be stopped by the primary pectus bar; and
a locking groove provided by being depressed downward from an upper surface of the locking body at the upper end thereof, and formed in a polygonal shape corresponding to a polygonal ratchet.

6. The orthopedic apparatus of claim 4, further comprising:
a stabilizer pressing and supporting the opposite ends of the primary pectus bar so as to prevent the primary pectus bar from rotating behind the sternum.

7. The orthopedic apparatus of claim 6, wherein
the stabilizer is provided at each of the opposite ends of the primary pectus bar and is perpendicularly coupled to the primary pectus bar.

8. The orthopedic apparatus of claim 6, wherein the stabilizer includes:
a connecting body supporting an outer surface of the primary pectus bar by coming into contact therewith;
an extended length portion formed by lengthening at each of opposite ends of the connecting body; and
a coupling protrusion formed by protruding inward from the extended length portion

9. The orthopedic apparatus of claim 8, wherein
the extended length portion is provided with a second through hole, and
the coupling protrusion is engaged with the notch groove so as to lock the primary pectus bar.

10. The orthopedic apparatus of claim 1, wherein
the primary pectus bar, the secondary pectus bar, and the locking member are made of a titanium alloy material.

11. An orthopedic apparatus for pectus excavatum, the orthopedic apparatus comprising:
a double pectus bar inserted behind and in front of a sternum to support the concave or to convex sternum; and
a locking means coupled to each of opposite ends of the double pectus bar and hooked to a rib, on which the opposite ends of the double pectus bar are disposed, to lock the double pectus bar.

12. The orthopedic apparatus of claim 11, wherein the double pectus bar includes:
a primary pectus bar inserted behind the sternum, and provided with a first coupling hole at each of opposite ends thereof;
a secondary pectus bar inserted in front of the sternum to be in parallel with the primary pectus bar, and provided with a second coupling hole at each of opposite ends thereof; and
a locking bolt locking the secondary pectus bar to the primary pectus bar by being coupled to both the second coupling hole and the first coupling hole.

13. The orthopedic apparatus of claim 12, wherein
the first coupling hole is in an annular shape with a thread corresponding to a thread of the locking bolt, and
the second coupling hole is in an oblong shape to allow adjustment of a position of the secondary pectus bar relative to the primary pectus bar.

14. The orthopedic apparatus of claim 12, wherein
the primary pectus bar is further provided with a locking hole at each of the opposite ends thereof, with the locking means coupled thereto.

15. The orthopedic apparatus of claim 14, wherein the locking hole includes:
an annular first locking hole; and
an oblong second locking hole communicating with the first locking hole, and having a width narrower than a diameter of the first locking hole.

16. The orthopedic apparatus of claim 15, wherein the locking means includes:
a hook hooked to the rib at a first end thereof; and
a fastening bolt engaged with a second end of the hook with a head thereof inserted through the first locking hole, and locked to the hook by the head being moved to the second locking hole.

17. The orthopedic apparatus of claim 16, wherein the locking means further includes:
a coupling slide provided with a through slot with the second end of the hook inserted thereinto so as to secure coupling between the hook and the fastening bolt, wherein a state where the second end of the hook is inserted into the through slot is fixed by the fastening bolt.

18. The orthopedic apparatus of claim 17, wherein
the hook is provided with a through hole at the second end thereof, and the coupling slide is provided with a communicating hole; and
the fastening bolt is engaged with both the communicating hole and the through hole to fix the state where the second end of the hook is inserted into the through slot of the coupling slide.

19. The orthopedic apparatus of claim 16, wherein
the hook is hook-shaped by the first end thereof being bent relative to the second end thereof to be in a C-shape, or is L-shaped by being bent at 90 degrees.

20. The orthopedic apparatus of claim 11, wherein
the double pectus bar and the locking means are made of a titanium alloy material.
